(19) United States
(12) Patent Application Publication
(10) Pub. No.: US 2006/0276639 A1
(43) Pub. Date: Dec. 7, 2006

(54) Conversion of sucralose-6-ester to sucralose

(75) Inventor: John Charles Fry, Broadbridge Heath (GB)

Correspondence Address: KOLISCH HARTWELL, P.C., 200 PACIFIC BUILDING, 520 SW YAMHILL STREET, PORTLAND, OR, 97204, US

(73) Assignee: Healthy Brands, LLC

(21) Appl. No.: 11/237,193

(22) Filed: Sep. 27, 2005

Related U.S. Application Data
(60) Provisional application No. 60/686,654, filed on Jun. 1, 2005.

(51) Int. Cl. C07H 3/04

(52) U.S. Cl. 536/123.13

(57) ABSTRACT

The present disclosure provides a process for producing sucralose from a feed mixture of (a) sucralose-6-ester, such as 6-O-acyl-4,1′,6′-trichloro-4,1′,6′-trideoxygalactosucrose, (b) salt including alkali metal or alkaline earth metal chloride, (c) water, and (d) other chlorinated sucrose by-products, in a reaction medium comprising a tertiary amide, wherein said process comprises deacylating, either before or after removal of the tertiary amide, the sucralose-6-ester under active cooling to produce an aqueous solution comprising sucralose, salt including alkali metal or alkaline earth metal chloride, and other chlorinated sucrose by-products. Additionally, the sucralose may be recovered from the aqueous solution, such as by extraction followed by crystallization or by extractive techniques alone.

### CROSS-REFERENCE

This application claims benefit to U.S. Provisional Application No. 60/686,654, which was filed on Jun. 1, 2005, the contents of which are incorporated herein by reference in the entirety for all purposes.

### FIELD

The present application is directed to methods and systems for the production of sucralose. More specifically, the present application is directed to methods and systems of converting sucralose-6-esters to sucralose.

### BACKGROUND

The artificial sweetener 4,1′,6′-trichloro-4,1′,6′-trideoxy-galactosucrose (“sucralose”) is derived from sucrose by replacing the hydroxyls in the 4, 1′ and 6′ positions with chlorine. In the process of making the compound, the stereo configuration at the 4 position is reversed. Therefore, sucralose is a galacto-sucrose having the following molecular structure:

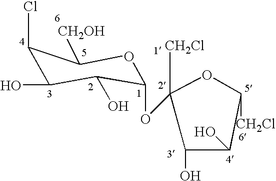

The direction of the chlorine atoms to only the desired positions is a major synthesis problem because the hydroxyls that are replaced are of differing reactivity; two are primary and one is secondary. The synthesis is further complicated by the fact that the primary hydroxyl in the 6 position is unsubstituted in the final product.

A number of different synthetic routes for the preparation of sucralose have been developed in which the reactive hydroxyl in the 6 position is blocked, as by an ester group, prior to the chlorination of the hydroxyls in the 4, 1′ and 6′ positions, followed by hydrolysis to remove the ester substituent to produce sucralose. Several of these synthesis routes involve tin-mediated synthesis of sucrose-6-esters. Illustrative are the tin-mediated routes disclosed by Navia (U.S. Pat. No. 4,950,746), Neiditch et al. (U.S. Pat. No. 5,023,329), Walkup et al. (U.S. Pat. No. 5,089,608—“Walkup-I”), Vernon et al. (U.S. Pat. No. 5,034,551), and Sankey et al. (U.S. Pat. No. 5,470,969), each of which is incorporated herein by reference in its entirety for all purposes.

The sucrose-6-esters produced by the above-cited synthesis routes are typically chlorinated by the process of Walkup et al., (U.S. Pat. No. 4,980,463—Walkup-II”), which is incorporated herein by reference in its entirety for all purposes. Other chlorination processes are available and effective for chlorinating the sucrose-6-esters. The chlorination process produces as a product a sucralose-6-ester, such as 4,1′,6′-trichloro-4,1′,6′-trideoxygalactosucrose-6-acetate, in solution in a tertiary amide, typically N,N-dimethylformamide (“DMF”), plus salts (produced as a result of neutralizing the chlorinating agent after completion of the chlorination reaction), chlorination reaction byproducts, and other impurities. Exemplary chlorination reaction byproducts include chlorinated carbohydrates other than sucralose, such as mono- and di-chlorinated sucrose, as well as other forms of chlorinated sucrose.

In previous processes, such as the processes disclosed in Walkup-II and the process disclosed in Navia et al. (U.S. Pat. No. 5,530,106—“Navia '106”), which is also incorporated herein by reference in its entirety for all purposes, sucralose is produced from the chlorination reaction mixture of Walkup-II by the following procedure:

a. after the neutralization step, the tertiary amide reaction vehicle for the chlorination reaction is removed, as by steam distillation (disclosed in Navia '106), which forms an aqueous mixture containing salts, sucralose-6-ester, chlorination byproducts, and other impurities (summarized as “tertiary amide removal step”);

b. the sucralose-6-ester is then recovered from the aqueous mixture by extraction using a suitable organic solvent, such as ethyl acetate (summarized as “sucralose-6-ester recovery step”);

c. the crude sucralose-6-ester is then de-acylated to form sucralose and deacylation byproducts (summarized as “de-acylation step”); and

d. the sucralose is recovered by counter-current extraction and purified by crystallization (summarized as “sucralose recovery step”).

In Navia et al. (U.S. Pat. No. 5,498,709, hereinafter “Navia '709”), which is incorporated herein by reference in its entirety for all purposes, a process is disclosed whereby the sucralose-6-ester is de-acylated directly (i.e., without the sucralose-6-ester recovery step), to produce an aqueous solution of sucralose, salts, chlorination byproducts, deacylation byproducts, and impurities, from which sucralose is recovered, as by extracting with an organic solvent, and the sucralose is then purified by counter-current extraction, crystallization or a combination of both techniques. Navia '709 disclosed two processes for de-acylating sucralose-6-ester and recovering the resulting sucralose. In the first process, the sucralose-6-ester was de-acylated in a tertiary amide solution and the sucralose was separated, or otherwise recovered, from the solution. In the second process, disclosed as being the more preferred process, Navia '709 described removing the tertiary amide prior to de-acylation and de-acylating the sucralose-6-ester in the aqueous mixture. Navia '709's disclosure effectively eliminated the sucralose-6-ester recovery step, requiring only the tertiary amide removal step, the de-acylation step, and the sucralose recovery step.

Chlorination byproducts and deacylation byproducts have been discussed briefly above. Speaking generally, chlorination byproducts include products of the chlorination reaction other than the desired sucralose-6-ester, such as mono- and di-chlorinated sucrose or other variations of the sucrose molecule wherein chlorine has not replaced the hydroxyls in the 4, 1′, and 6′ positions or has replaced the hydroxyls in additional positions. Similarly, deacylation byproducts include products of the deacylation reaction other than sucralose. As used herein, the term sucralose refers to the 4,1′,6′-trichloro-4,1′,6′-trideoxygalactosucrose molecule introduced above. Exemplary, but not limiting, deacylation byproducts include breakdown products of sucralose, such as 3′,6′ anhydro-sucralose and other chlorinated carbohydrates.

Navia '709 described processes whereby sucralose-6-ester was de-acylated while in a tertiary amide solution or after removal of the tertiary amide. In both processes, Navia '709 disclosed adding sufficient aqueous alkali to the chlorination product (or the chlorination product after removal of the tertiary amide) to attain a pH of 11 (±1) and maintaining that pH for sufficient time to remove the 6-acyl function and to produce sucralose. As described in Navia '709, the de-acylation step generally required between 30 minutes and 2 hours. Navia '709 discusses the deacylation reaction temperature only superficially, describing the temperature at the start of the deacylation reaction and that the reaction proceeds at ambient temperature with no temperature control during the deacylation reaction. Due to the heat released upon addition of the alkali material, it is believed that the reaction temperature of Navia '709's deacylation step ranges from 15° C. to 35° C. It is further believed that the reaction temperature rises rapidly upon addition of the base and gradually cools due to interaction with the cooler ambient air.

As discussed briefly above, Navia '709 disclosed that deacylation prior to removal of the tertiary amide was strongly disfavored. Navia '709 disclosed that the direct de-acylation in the presence of the tertiary amide, even at a pH of 11±1, was carried out at the expense of some tertiary amide, which was lost by caustic hydrolysis to dimethylamine and sodium formate. The loss of the tertiary amide reduced the recycle efficiencies of the tertiary amide. Moreover, the presence of the tertiary amide hydrolysis products and sucralose in solution was disclosed as complicating the sucralose recovery process.

### SUMMARY

The present disclosure provides a method for deacylating sucralose-6-ester to produce sucralose from a feed mixture of (a) sucralose-6-ester, such as 6-O-acyl-4,1′,6′-trichloro-4,1′,6′-trideoxygalactosucrose, (b) salt including alkali metal or alkaline earth metal chloride, (c) water, and (d) other chlorination byproducts, in a reaction medium. The method includes deacylating, either before or after removal of the tertiary amide, the sucralose-6-ester by raising the pH of the deacylation reaction medium to a pH of at least about 11 while actively cooling the reaction medium to maintain the temperature of the reaction medium in a predetermined temperature range. Raising the pH to at least about 11 while actively cooling the reaction medium initiates the deacylation reaction to produce a solution comprising sucralose, salts, water, chlorination byproducts, and deacylation byproducts. The method further includes neutralizing the pH of the reaction medium.

### DETAILED DESCRIPTION

Methods of deacylating sucralose-6-ester to produce sucralose from a feed mixture of (a) sucralose-6-ester, such as 6-O-acyl-4,1′,6′-trichloro-4,1′,6′-trideoxygalactosucrose, (b) salts, including alkali metal or alkaline earth metal chloride, (c) water, and (d) other chlorination byproducts in a reaction medium may include raising the pH of the reaction medium of (a), (b), (c) and (d) to at least about 11 while actively cooling the solution to a predetermined temperature range, such as from about 0° C. to about 25° C. for a period of time sufficient to effect said deacylation, to produce an aqueous solution comprising sucralose, salts, chlorination byproducts, and deacylation byproducts in a reaction medium. The reaction medium may then be neutralized and the sucralose recovered from the solution.

The feed mixture may be produced through a variety of suitable procedures. Generally, the feed mixture may be produced by esterifying sucrose to produce sucrose-6-ester, chlorinating the sucrose-6-ester to produce sucralose-6-ester, and quenching the chlorination reaction to produce the feed composition of sucralose-6 -ester in a tertiary amide reaction medium. The quenching of the chlorination reaction product generally results in a solution having a pH ranging from about 5 to about 7. When sodium hydroxide is used in the quench step and the tertiary amide is DMF, the salts that are formed in the quench step may include sodium chloride, dimethylamine hydrochloride and small amounts of sodium formate. Exemplary, but not limiting, methods of producing sucralose-6-ester were described above, including the methods of Walkup-II and Navia '106.

The methods of the present disclosure may employ as the feed mixture a composition comprising 6-O-acyl-4,1′,6′-trichloro-4,1′,6′-trideoxygalactosucrose (sucralose-6-ester) in a tertiary amide (preferably DMF) reaction medium, such as the neutralized (quenched) product of the chlorination reaction described by Walkup-II, cited above. Exemplary 6-O-acyl-4,1′,6′-trichloro-4,1′,6′-trideoxygalactosucrose esters may include 6-O-acetyl-4,1′,6′-trichloro-4,1′,6′-trideoxygalactosucrose (sucralose-6-acetate) and 6-O-benzoyl-4,1′,6′-trichloro-4,1′,6′-trideoxygalactosucrose (sucralose-6-benzoate). Other suitable sucralose-6-esters may also be used.

As described above, the reaction medium of the feed mixture generally includes a tertiary amide, such as DMF, from prior steps in the production of the sucralose-6-ester. The methods of the present disclosure may include deacylating the sucralose-6-ester in the presence of the tertiary amide. The tertiary amide may then be removed, such as by steam distillation or by extraction, and the sucralose recovered, such as by extraction followed by crystallization or by extractive techniques alone. Alternatively, the tertiary amide, or the majority thereof, may be removed prior to initiating the deacylation reaction of the present disclosure. The tertiary amide may be removed by any suitable process, such as by steam distillation. The methods of the present disclosure result in a deacylation reaction under controlled temperatures that does not produce tertiary amide hydrolysis products to any significant degree. Accordingly, the recycle efficiency of the tertiary amide is not reduced by deacylating prior to removal of the tertiary amide. Additionally, the recovery of the sucralose is not further complicated by the presence of tertiary amide hydrolysis products.

Whether the tertiary amide is removed before or after the deacylation reaction, the tertiary amide, or at least a major proportion thereof, may be removed, either from the quenched feed mixture or the deacylated reaction medium, through steam stripping operations or other separation procedures. In some aspects of the present disclosure, at least 95%, and preferably, from about 98 to 99.9%, of the tertiary amide may be removed. Upon removal of the DMF (or other tertiary amide) by steam stripping or otherwise, the DMF is effectively replaced with water in the process stream and the DMF can be recovered from the aqueous overheads by distillation and can be recycled.

A number of industrial-scale and laboratory scale processes may be used to remove the tertiary amide from the reaction medium, whether from the quenched feed mixture or from the deacylated sucralose composition. By way of example and not limitation, an example of a laboratory-scale, falling-film, packed-column, steam distillation apparatus designed for stripping the DMF from quenched sucralose-6-ester chlorination products, the feed mixture, is a 5.0-cm diameter, 90-cm long vacuum-jacketed distillation column packed with 5-mm Raschig rings or other suitable packing. Additionally or alternatively, a 15-plate, jacketed, Oldershaw column may be used. The quenched product, which is typically preheated, is introduced into the top of the column at a rate of about 5.0-5.5 grams per minute. Steam is introduced into the column through a sidearm located at the bottom of the column. As condensate-free steam is required, the steam is passed through a “preboiler” to trap any condensate carried over. In the laboratory, this preboiler is typically a small multineck flask fitted with a heating mantle. Typical steam feed rates are in the range of 38-47 grams per minute (calculated by adding the weights of overhead and bottom products, and then subtracting the weight of chlorination feed), which corresponds to a steam-to-feed ratio ranging from 4:1 to 12:1, with steam to feed ratios of between 7.5:1 and 9:1 being typical for the packed column assembly. An exemplary embodiment would use more plates with a lower steam/feed ratio, e.g., 15 plates with a steam/feed ratio of about 4:1.

The preheating of the quenched chlorination feed before it is introduced into the top of the column is conducted in order to increase the efficiency of the stripping operation. Preheating is typically conducted in the laboratory by passing the feed through an enclosed glass coil apparatus heated with a secondary source of steam. The feed is normally heated to about 90°-95° C. The efficiency of DMF removal can also be enhanced by employing a “reboiler” (i.e., by heating the bottoms product in such a way that it refluxes up into the stripping column).

Temperatures may be measured at least at two places on the apparatus using thermocouple devices or other techniques. In addition to the quenched chlorination feed temperature described above, the temperature of the vapors passing through the distillation column head also may be measured. Head vapor temperatures are typically in the range of from about 99° C. to about 104° C.

A typical quenched chlorination product of sucrose-6-acetate, or feed mixture, contains about 1.5-5 wt % sucralose-6-ester, about 35-45 wt % DMF, about 35-45 wt % water, and about 12-18 wt % salts. After passage of such feed mixture through the laboratory-scale steam stripping apparatus, bottoms products will typically consist of about 1-3 wt % sucralose-6-ester, about 0.1-0.5 wt % DMF, about 80-90 wt % water, and about 8-12 wt % salts (expressed as NaCl, based on sodium and chloride assays). Under typical laboratory conditions, which involve a column residence time of 7-10 minutes, no decomposition of sucralose-6-ester is detectable, provided the pH of the quenched chlorination feed is neutral to slightly acidic (pH 5.0-7.0).

Similar conditions can be used for steam-stripping the DMF from a quenched and deacylated reaction medium. It is within the scope of the present disclosure that other tertiary amide removal techniques may be used. The steam stripping and specific apparatus and procedures described above are by way of example only and not limitation.

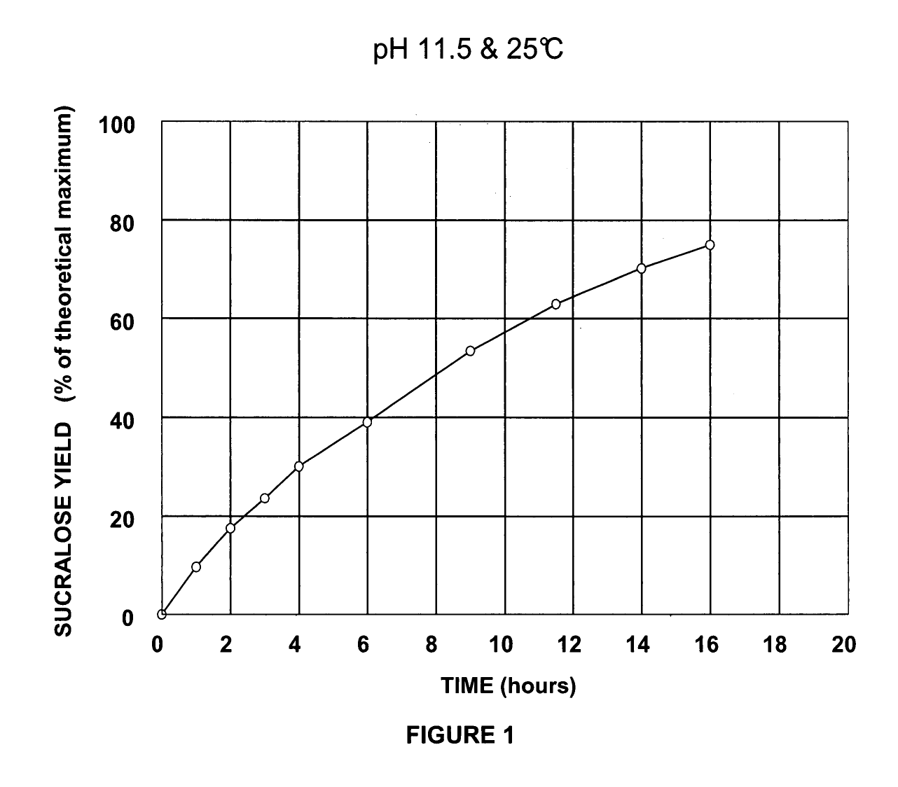

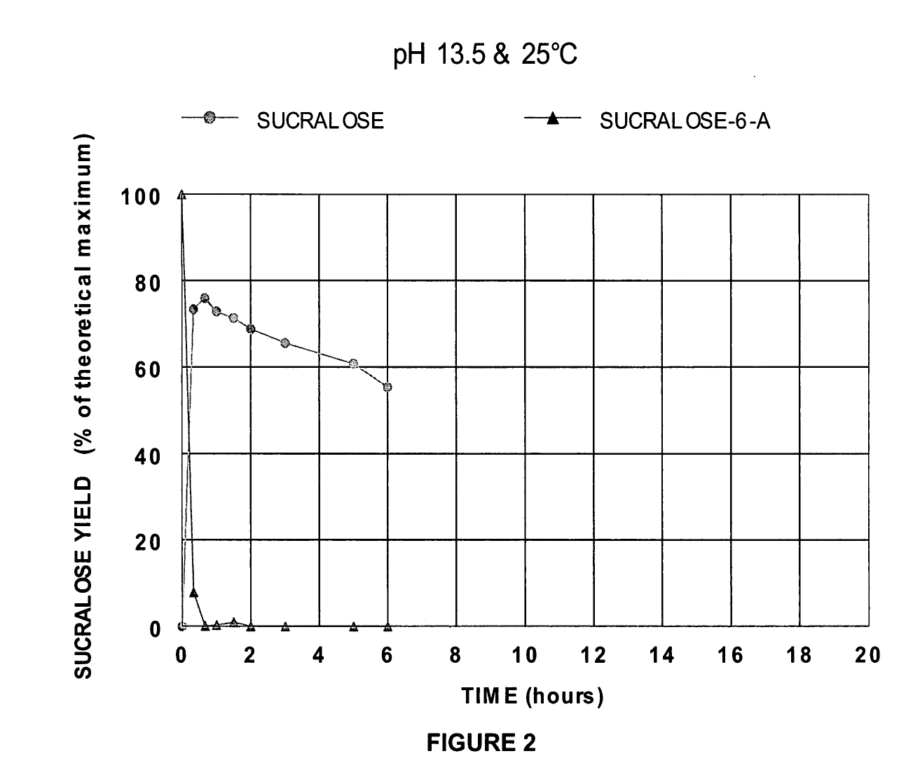

Whether the feed mixtures for the deacylation methods are in a tertiary amide reaction medium or the tertiary amide has been removed from the solution, the present deacylation methods deacylate the sucralose-6-ester under controlled conditions to produce sucralose with increased sucralose yields, with delayed onset of degradative reactions that reduce the sucralose yield, and/or with decreased production of deacylation byproducts, as compared to prior deacylation techniques. As indicated above, prior deacylation procedures were limited to a pH of about 11±1 and were conducted at room temperature. Due to the rapid temperature increase at the beginning of the deacylation reaction, caused by the addition of the basic material, it is believed that in reactions with no temperature control the reaction temperature increases to at least about 35° C. Due to interactions with the ambient air at room temperature, the reaction temperature then decreases over time to yield an estimated average temperature of at least 25° C. over the course of the reaction. FIG. 1 illustrates the sucralose yield as a function of time for a deacylation reaction under controlled conditions of a pH of 11.5 and a temperature of 25° C. By comparison, FIG. 2 illustrates the sucralose yield of a deacylation reaction under controlled conditions having a pH of 13.5 and a temperature of 25° C.

Each of FIGS. 1-5 represent sucralose yields from deacylation reactions carried out under controlled conditions of pH and temperature. The details of each reaction are provided below as Examples. The details of the pH and temperature control are similarly provided below. Any suitable process may be used to control the pH and the temperature to an acceptable degree. As will be seen, there is a balance between pH and temperature such that greater control over the temperature may allow less control over the pH. Similarly, less control over the temperature may require greater control over the pH of the deacylation reaction. In some aspects of the present methods, such as the Examples described below and shown in FIGS. 1-5, the pH is controlled to within 0.05 of a pH set point and the temperature is controlled to within 0.1° C. of a temperature set point.

In industrial applications of the present methods, different levels of control may be implemented. For example, the average temperature of the reaction vessel, in a batch reaction, or the temperature at a particular location in the reactor, such as in continuous flow reactors, may be controlled to within 1.0° C. of the temperature set point. Similarly, the pH may be controlled to within 0.5 of the pH set point. As can be appreciated, accurate measurement of the pH of a reaction medium can be complicated when the composition being measured includes a mixed aqueous/non-aqueous solvent, such as water and the tertiary amide DMF. As used herein, and in the methods described herein, the pH measured is strictly the _{w}^{s}pH. Accordingly, the pH may be measured by calibrating the measuring cell in aqueous buffers, as is customary, measuring the pH of the mixed solvent, and recording the pH reading without further adjustment or calibration.

As can be seen in FIGS. 1 and 2, increasing the pH of the deacylation reaction significantly increases the reaction rate producing a maximum yield after about 1 hour, as compared to 16 or more hours at the lower pH. While increasing the reaction rate is generally desirable in industrial processes to reduce operating costs, FIG. 2 illustrates that if the reaction proceeds too far, the sucralose yield is actually reduced. Without being bound by theory, the reduced sucralose yield beyond the peak yield time is believed to be due to the breakdown of sucralose at high pH to various deacylation byproducts, including 3′,6′ anhydro-sucralose. The point of maximum yield, or peak yield time, is relatively short-lived in FIG. 2, which complicates industrial or laboratory processes designed to maximize the yield of sucralose. Generally, the reaction progress is monitored by periodic sampling and analysis to determine when the reaction is at its peak yield. The analytical procedures alone can take from tens of minutes up to an hour or more to complete. Accordingly, a deacylation reaction with a peak yield time shorter than 30 minutes or so is inconveniently short due to the difficulty in identifying the point of maximum yield and stopping the reaction before the yield is reduced.

A comparison of FIGS. 1 and 2 suggest then that the pH should be carefully controlled to reduce the conversion of the desired sucralose to the undesired deacylation byproducts. Accordingly, various methods of stabilizing the pH of the deacylation reaction in a pH range from 8.0 to 12.0 have been disclosed, including the addition of buffering agents to the quenched chlorination reaction medium to buffer the deacylation reaction, such as disclosed by Vernon et al. (U.S. Pat. No. 6,890,581). However, the addition of buffering agents to the deacylation reaction further dilutes the sucralose and complicates the recovery of sucralose.

Conversion of sucralose-6-ester to sucralose according to the present disclosure can be carried out over a larger pH range, without the need for buffering agents, by actively cooling the deacylation reaction solution. Active cooling during the deacylation reaction enables deacylation over a greater pH range, including a higher pH range, and greater conversion of sucralose-6-ester to sucralose while also limiting the conversion to other undesirable chlorinated sugars, such as deacylation byproducts, and limiting the breakdown of the tertiary amide.

The feed mixture (such as quenched chlorination products before or after removal of the tertiary amide) may be raised to a pH of at least about 11 in a temperature controlled environment to effect deacylation and production of sucralose. The methods of the present disclosure provide a deacylation reaction with a reaction rate controlled by both the pH of the reaction medium and the temperature of the reaction medium. As discussed below, an elevated pH increases the rate of the deacylation reaction and the rate at which the sucralose is converted to the various deacylation byproducts. Additionally, the active cooling is shown below to limit the deacylation reaction rate and also to prolong the peak yield time by delaying the conversion of sucralose to deacylation byproducts.

In a laboratory setting, the temperature may be controlled via active cooling in a number of methods, such as via an ice bath or other conventional cooling techniques. Similarly, in larger scale applications, the active cooling during the deacylation reaction may be provided by co-current or counter-current flow systems or other conventional heat exchange or temperature control systems. In one exemplary configuration, a jacketed reaction vessel having a jacket at least partially surrounding a reaction cavity. The temperature of the jacket may be controlled to actively cool the reaction medium to a predetermined temperature range. The temperature of the jacket may be controlled by circulating a heat transfer fluid through the jacket. Additionally or alternatively, other heat exchange or temperature control components or features may be employed. In either scenario, the active cooling systems may be configured to maintain the deacylation reaction in a predetermined temperature range. In the absence of the active cooling system, the temperature of the deacylation reaction solution may be driven upwards by a number of factors including the ambient environment (i.e., interaction of the reaction solution with the room temperature air around it) and/or the heat of reaction of one or more chemical reactions occurring within the reaction solution, such as acid/base reactions, etc. Depending on the reaction conditions, such as composition of the feed mixture and composition of the base used to raise the pH, the active cooling systems may be required to provide more or less cooling effect to maintain the temperature of the reaction medium within the predetermined range.

The actual amount of cooling provided in a particular deacylation reaction may be based at least in part on the starting conditions of the reaction; the estimated or actual heat of reaction for the one or more reactions in solution; the estimated, average, or actual pH of the reaction before or during the reaction; the ambient temperature; the amount of time desired for completion of the reaction; and/or the desired level of sucralose conversion and/or purity. Other factors may also influence the amount of active cooling required by the methods of the present disclosure. Suitable heat exchange systems, or active cooling systems, may be configured to maintain the desired reaction temperature during the course of the reaction, whether the deacylation is carried out as a batch reaction or a continuous reaction.

Upon initiation of the deacylation reaction, the amount of heat added to the reaction solution (from the ambient environment, from one or more reactions, or from other sources) may vary over time as the reaction progresses. Accordingly, in some aspects of the present disclosure, the amount of active cooling provided may be related to the pH of the solution and/or the progress of the deacylation reaction. In other embodiments, the amount of active cooling provided to the deacylation reaction solution may vary over time according to a predetermined pattern or model. In still other embodiments, the amount of active cooling provided may be constant throughout the deacylation reaction. Depending on the mode of the deacylation reaction, batch process or continuous flow process, the active cooling system may be modified appropriately to enable manual or automatic control over the amount of active cooling provided to reaction medium, such as by increasing or decreasing the flow rate of one or more streams or by providing additional coolant, to maintain the desired reaction temperature.

It is within the scope of the present disclosure that the pH of the solution and the active cooling of the solution may be balanced to provide optimum reaction conditions. Various systems and methods are within the scope of the present disclosure for measuring and/or controlling the pH of the solution and/or the amount of active cooling provided to the solution. Exemplary, but not limiting systems, are described herein.

For example, the temperature can be measured either of the deacylation reaction medium or of the heat exchange fluid used to cool the reaction medium. Changes in temperature in the reaction medium and/or the heat exchange fluid may be monitored and correlated to determine the rate of reaction, the pH of the reaction solution, and/or the amount of cooling required. Additionally or alternatively, the pH of the reaction solution may be measured, either continuously or periodically, which may be used at least in part to determine the active cooling needs of the reaction system and/or the required pH adjustment. Moreover, both the pH and the temperature may be monitored directly with the results used to control the amount of active cooling provided and to control the amount of acid and/or base added to the reaction medium to maintain the reaction medium within a predetermined range of the temperature and pH set-points, respectively.

Additionally or alternatively, the temperature may be controlled based on measured temperature alone, such that the temperature is maintained within a predetermined range, which may be established by the target pH of the reaction. For example, the deacylation reaction may be run in a predetermined temperature range, such as from about 0° C. to about 25° C., and the active cooling applied to keep the temperature of the reaction medium in the predetermined range. Moreover, the active cooling may be applied to keep the temperature of the reaction medium within a predetermined margin from a temperature set point. As discussed herein, the desired predetermined temperature range and/or temperature set point may depend on a number of factors, such as the desired rate of reaction, the desired duration of the peak yield time, the desired purity, and the projected or target pH of the reaction.

It is within the scope of the present disclosure that a single target reaction temperature or temperature range can be maintained via active cooling for a number of reaction pH conditions. For example, an actively cooled reaction maintained at about 17.5° C. may be appropriate for reactions at a pH ranging from about 12 to about 14. However, a relatively lower reaction temperature combined with a relatively lower pH may slow the deacylation reaction to a point that the reaction takes too long to be practically or commercially reasonable. Similarly, a relatively higher reaction temperature combined with a relatively higher pH may shorten the peak yield time to reduce the recoverable sucralose yield. The balance between the temperature of the reaction and the pH of the reaction may vary depending on a number of circumstances as discussed herein and the selection of an appropriate balance is within the scope of the present disclosure. Exemplary combinations of pH and temperature are described herein and other suitable combinations may be implemented.

As an illustrative example, a lower temperature range, such as between about 5° C. and about 10° C., may be selected for a deacylation reaction occurring at a higher pH range, such as between about 13.5 and about 14.0. Similarly, and as a non-limiting example only, a relatively higher temperature range, such as between about 17.5° C. and about 25° C., may be selected for a deacylation reaction occurring at a relatively lower pH range, such as between about 12.0 and about 13.5.

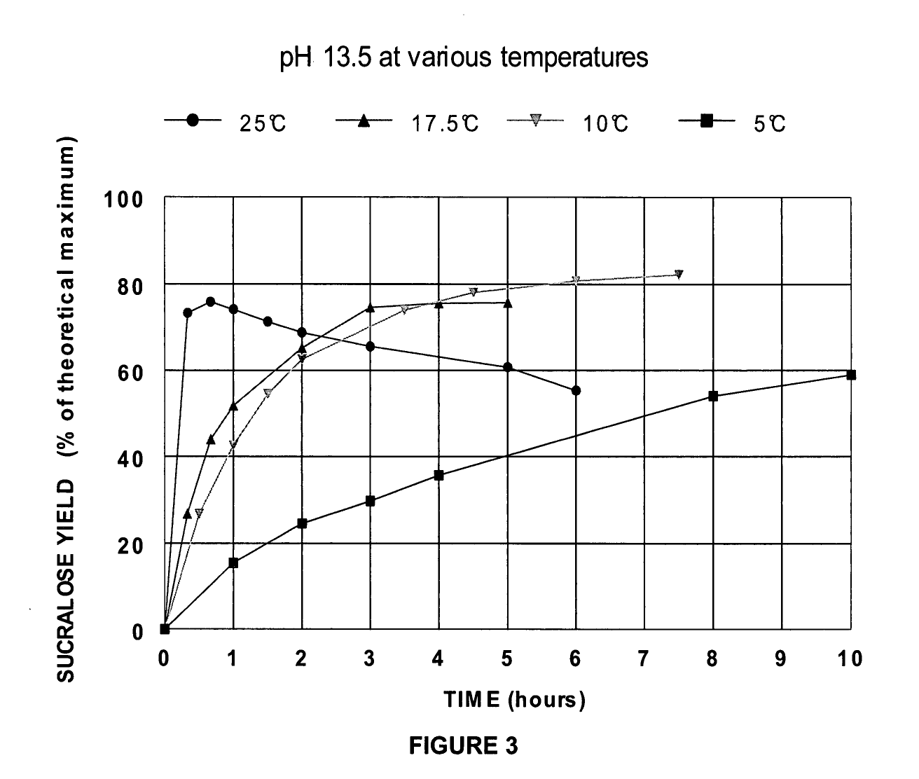

FIG. 3 illustrates the reactions of Examples 2 and 3 discussed in detail below. Generally, FIG. 3 illustrates the sucralose yield as a function of time for a deacylation reaction carried out in a reaction medium having a pH of about 13.5 and at various temperatures. FIG. 3 illustrates that active cooling renders the reaction more controllable. With decreasing reaction temperatures, the reaction is slowed so that the maximum yield is reached over a longer period of time. Accordingly, the peak yield time is prolonged, which facilitates the identification of the maximum yield and the control of the reaction to recover the maximum yield rather than some amount less than the maximum yield.

FIG. 3 also illustrates that active cooling to 10° C. increases the yield of sucralose over the yields at other temperatures and over the yields at 25° C. and a pH of 11.5 (illustrated in FIG. 1). Without being bound by theory, it is presently believed that the yield is increased because the degradative reactions are relatively more inhibited by the cool conditions than is the desired deacylation reaction, which allows more sucralose to be produced and to survive intact without leading to the undesired deacylation byproducts. It is possible that the reaction at 5° C. would provide still higher yield than the yield at 10° C. While it may be possible to extend the reaction longer than the 10 hours shown in FIG. 3, such a prolonged reaction time is generally undesirable on an industrial scale, due to slow throughput and/or the required extra capital expenditure to provide the increased capacity necessary to accommodate the extended reaction time.

It is apparent, however, that the combination of high pH and active cooling permits a substantial degree of control over the reaction time to reach the maximum yield. Additionally, the peak yield time may be prolonged to facilitate the identification of when the reaction is at a point of maximum sucralose yield. Accordingly, a choice of optimum conditions for any particular purpose may be made in light of the economic balance between achieving the maximum yield of which the reaction is capable, by the lower reaction temperature, and the cost of providing the necessary equipment and reaction time required by the lower reaction temperature. It is possible that a marginally lower than maximum yield, such as the yield obtained at 17.5° C., may be economically preferred over the longer reaction times of 10° C. or 5° C. Depending on the market value of sucralose, however, it may alternatively be economically preferred to extend the reaction time by lowering the reaction temperature to 5° C. or even lower to obtain the highest possible sucralose yield.

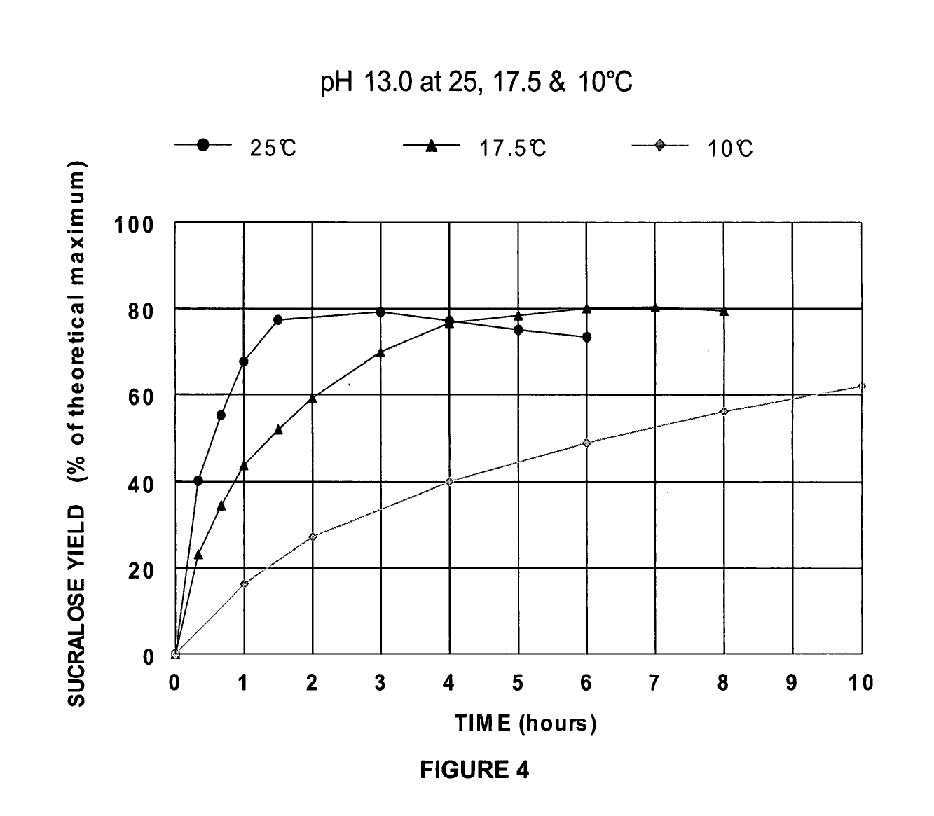

The deacylation reactions were also conducted at a pH of about 13.0 and at a variety of temperatures. The results are shown in FIG. 4. As illustrated, lowering the pH of the reaction slowed the reaction as expected. However, when the reaction is conducted at 25° C., the peak yield time is still relatively short as shown by the yield beginning to decline immediately after the 3 hour point. Accordingly, the application of active cooling is preferred in order to prolong the peak yield time wherein the reaction is maintained in the vicinity of the maximum yield. As used herein, peak yield time refers to the amount of time during which the sucralose yield of the reaction is substantially close to the maximum yield obtainable before the yield begins to decrease, such as within 5% of the maximum yield. On an industrial scale, prolonging the peak yield time would give a better chance of stopping the reaction at the optimum time. As illustrated in FIG. 4, cooling the reaction to 17.5° C. prolongs the peak yield time as compared to the reaction at 25° C. As further illustrated in FIG. 4, actively cooling the reaction to about 10° C. with a reaction pH of about 13.0 significantly slows the reaction, similar to running the reaction at 5° C. with a pH of about 13.5. As described above, the reaction temperature and pH conditions may be adapted or customized to obtain the economically desired yield in an economically desired reaction time.

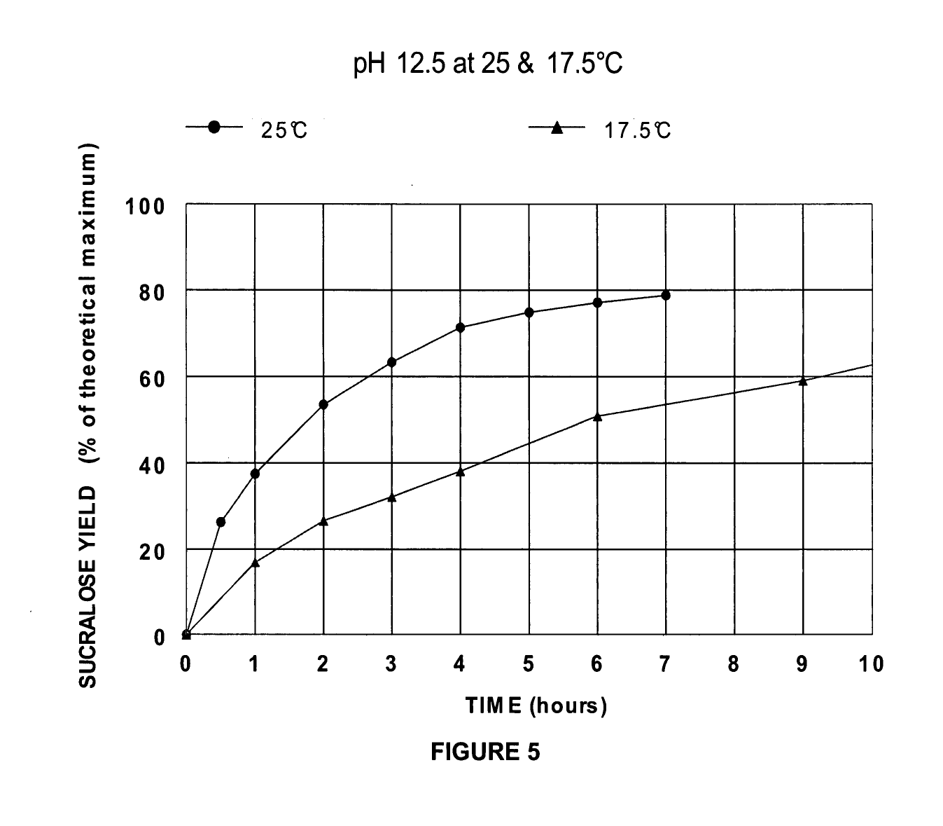

To further illustrate the relationship between temperature and pH in deacylation reactions, deacylation reactions were performed at a pH of about 12.5 and two different temperatures. The results are illustrated in FIG. 5. As can be seen, the trend towards slowed reaction times and prolonged peak yield times is continued. However, as with the lower temperatures at the higher pH's, the deacylation reactions at a pH of 12.5 proceeded slowly at a temperature of 17.5° C. and of 25° C.

FIGS. 3, 4, and 5 together illustrate that there is an interplay between pH and temperature that permits fine control over the course of the deacylation reaction and, in particular, the time to maximum yield as well as the duration of the peak yield time. This interplay is exemplified by the almost identical courses exhibited by reactions under conditions as varied as pH 13.5 at 5° C., pH 13.0 at 10° C. and pH 12.5 at 17.5° C.

It is thus possible to choose a variety of pH and temperature conditions to achieve a convenient time to maximum yield. However, there is a preference for conditions that simultaneously maximize the yield of sucralose, prolong the peak yield time to facilitate stopping the reaction at the optimum moment, and ensure that the reaction is sufficiently rapid that it can be completed without undue delay. Exemplary, but not limiting, combinations of pH and temperature are illustrated for reaction conditions such as a pH of 13.5 at 10° C. or a pH of 13.0 at 17.5° C.

Additionally or alternatively, methods within the scope of the present disclosure may include varying the reaction conditions over the course of the reaction. For example, a relatively high pH may be combined with a relatively high temperature during the initial stages of the reaction followed by modifying the temperature and/or the pH to prolong the peak yield time. For example, the reaction could be initiated at a pH of 13.5 and a temperature of 17.5° C. for the first 2-3 hours of the reaction before the temperature is decreased through active cooling to 10° C. Such a change in the reaction conditions may successfully accelerate the deacylation to produce sucralose (while at the higher temperature) while also successfully delaying the degradative reactions that decrease the maximum yield and shorten the peak yield time. Various combinations of temperature and pH for varying amounts of time may be implemented to achieve the highest maximum yield in an economically viable reaction time.

The above described active cooling systems and methods may be applied to an otherwise standard deacylation reaction wherein the sucralose-6-ester is deacylated by increasing the pH of the reaction, such as suggested in the above description. Whether before or after removal of the tertiary amide, the sucralose-6-ester may be deacylated by increasing the pH of the reaction medium to a pH of at least about 11 while actively cooling the reaction medium, as described above, for a period of time sufficient to effect the deacylation. This step is typically carried by adding sufficient alkali metal hydroxide, such as sodium hydroxide, with agitation, to increase the pH to the desired level. In some applications, the pH may range between about 12 and about 14. Additionally or alternatively, the pH may range between about 12.5 and about 13.5.

Reaction temperatures between about 0° C. and about 25° C. have been found to be useful with temperatures between about 5° C. and about 17.5° C. being more useful and temperatures between about 10° C. and about 17.5° C. being more useful. The more useful reaction temperature may vary depending on the reaction pH, as described in more detail above. Reaction time will vary depending on the reaction pH and the reaction temperature. However, in temperature-pH balanced deacylation reactions within the scope of the present disclosure, the reaction times may be as short as 30 minutes and as long as 48 hours. Reaction temperatures between about 5° C. and about 25° C. and pH between about 12.5 and about 13.5 have been found to be useful with reaction times between about 30 minutes and about 24 hours, depending on the desired sucralose yield and the desired duration of the peak yield time.

At the conclusion of the deacylation, the base present will normally be neutralized, as by addition of hydrochloric acid, to a pH of about 5 to 7. After the neutralization, the aqueous reaction medium contains sucralose, salts (as above, plus the salt produced by the neutralization step described immediately above), and other chlorinated sucrose byproducts, such as the chlorination byproducts and the deacylation byproducts.

Following the deacylation reaction and neutralization, the sucralose may be isolated by extraction of the aqueous brine solution with a variety of organic solvents. These solvents include methyl acetate, ethyl acetate, methyl ethyl ketone, methyl iso-butyl ketone, methyl iso-amyl ketone, methylene chloride, chloroform, diethyl ether, methyl tert-butyl ether, and the like. A preferred solvent, for reasons of extraction selectivity, ease of recycle, and toxicological safety, is ethyl acetate.

In the laboratory, sucralose isolation is typically conducted by first partially evaporating the crude neutralized deacylation reaction product. About half the water present may optionally be removed, producing a solution containing about 2-5 wt % carbohydrates and about 15-25 wt % salts. Isolation is normally conducted by carrying out three sequential extractions with ethyl acetate or other appropriate solvent. The extracts are combined, and may optionally be washed with water (to partially remove any residual DMF and dichlorodideoxysucrose derivatives, which to some extent are partitioned into the organic phase).

In addition to the batch extraction technique outlined above, extraction may also be carried out continuously on the dilute (not concentrated by evaporation) stream in a counter current mixer/settler extraction system. The advantage is that no prior evaporation-concentration step is required. Various suitable counter-current extraction techniques are known in the art as well as other suitable extraction techniques.

Once the crude sucralose has been recovered from the aqueous brine as a solution in an appropriate organic solvent, it is concentrated and the product can be purified by crystallization and recrystallization from the same solvent until the required purity is achieved. Alternatively, the sucralose may be crystallized from a solvent mixture such as methanol-ethyl acetate or from water to achieve the desired purity level. Sequential partitioning of the sucralose between solvent-water mixtures in a counter-current manner also allows a purification to be achieved and likewise opens the possibility of a direct liquid fill process (i.e., no material isolation needed; the final process stream having the requisite specifications to be directly packaged for use).

Another noteworthy aspect of the purification/recovery process described above (that is, extraction followed by crystallization) is that the same solvent can be used for extraction and the purification step. Typically (i.e., with other chemical materials), it is rare that the chemical product to be purified will crystallize from the same solvent that is used to extract it. In the present case, however, a combination of dilution and relatively low levels of impurities allows the sucralose to remain in solution during the extraction, and then after the solution containing the extracted sucralose is concentrated, the sucralose product can then be crystallized from the same solvent.

The sucralose produced by the deacylation reaction under active cooling may be isolated in a number of processes. Additionally, due to the increased yield, higher purity, and decreased concentrations of other chlorinated carbohydrates, novel processes may be developed for isolation of the sucralose. Additionally or alternatively, known processes used in other contexts may be found to be effective for the isolation of sucralose produced according to the deacylation reaction with active cooling as described herein. It is within the scope of the present disclosure that any one or more of these various isolation techniques may be used to isolate sucralose converted from sucralose-6-ester according to the present disclosure.

### EXAMPLES

### Example 1

200 mg of sucralose-6-acetate was dissolved in 9 g of 50% w/w DMF/water to which was added 1 g (accurately weighed) of a stock solution of a chromatographic internal standard of sodium benzoate in 50% w/w DMF/water. The solution was contained in a glass jacketed vessel through the walls of which was circulated a heat transfer fluid pumped in closed circuit through a thermostatic controller accurate to ±0.1° C. The contents of the vessel were stirred throughout and both internal solution temperature and pH monitored. The solution temperature was adjusted to 25±0.1° C. and the pH was adjusted to 11.5±0.05 using 1 molal sodium hydroxide dissolved in 50% w/w DMF/water for crude adjustment and 0.1 molal sodium hydroxide dissolved in 50% W/W DMF/water for fine adjustment. The pH was monitored throughout the reaction and small additions of 0.1 molal sodium hydroxide dissolved in 50% w/w DMF/water were made to hold the pH constant at 11.5±0.05.

Samples were taken periodically and the reaction stopped by neutralizing the solution with 10% v/v acetic acid in water. Neutralized samples were stored at 0-5° C. until analyzed by HPLC to determine the amount of sucralose released. The results of the HPLC analysis are shown in FIG. 1 as the amount of sucralose released, expressed as a percentage of the maximum theoretical molar yield, as a function of time.

### Example 2

The procedures of Example 1 were repeated at pH 13.5 and both the yield of sucralose and the residual amount of sucralose-6-acetate were determined at various times by HPLC. The result is shown in FIG. 2, from which it can be seen that the deacylation occurs much more rapidly at pH 13.5 than at pH 11.5. It is also evident that the sucralose-6-acetate is rapidly exhausted, though not completely converted to sucralose, and that the sucralose yield reaches a maximum roughly coincident with the consumption of all the sucralose-6-acetate. After this maximum yield point, the yield of sucralose declines. The decreased sucralose yield is believed to be due to the breakdown of sucralose at high pH to various deacylation byproducts, including 3′,6′ anhydro-sucralose. When analyzed by HPLC, reaction products of this deacylation have been shown to include a substance with the same retention time as an authentic sample of 3′,6′ anhydro-sucralose.

### Example 3

The procedures of Example 2 were repeated except that the temperature of the reaction was held at 17.5±0.1° C. by means of circulating a cooled heat transfer liquid through the jacket of the reaction vessel. The yield of sucralose was determined at various times by HPLC. The procedures of Example 3 were further repeated at temperatures of 10±0.1° C. and 5±0.1° C. The results of the procedures of Examples 2 and 3 are shown plotted together in FIG. 3.

### Example 4

The procedures of Example 2 were repeated three times with the pH being adjusted to 13.0±0.05 and the temperature of the reaction being held at 25, 17.5, and 10° C. (each ±0.1° C.), respectively. The yield of sucralose was determined at various times by HPLC. The results are shown in FIG. 4.

### Example 5

The procedures of Example 2 were repeated twice with the pH being adjusted to 12.5±0.05 and the temperature of the reaction being held at 25 and 17.5° C. (each ±0.1° C.) respectively. The yield of sucralose was determined at various times by HPLC. The results are shown in FIG. 5.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring, nor excluding, two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower or equal in scope to the original claims, are also regarded as included within the subject matter of the inventions of the present disclosure.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

I claim:

1. A method of deacylating sucralose-6-ester in a feed mixture of (a) sucralose-6-ester, (b) salt including alkaline metal or alkaline earth metal chloride, (c) water, and (d) other chlorinated sucrose byproducts, in a reaction medium comprising a tertiary amide, the method comprising:
actively cooling the reaction medium to a predetermined temperature range;
raising the pH of the reaction medium to a pH of at least about 11 while continuing to actively cool the reaction medium to maintain the temperature of the reaction medium in the predetermined temperature range to initiate a deacylation reaction and to produce a solution comprising sucralose, salt including alkaline metal or alkaline earth metal chloride, water, and other chlorinated sucrose byproducts; and
neutralizing the pH of the reaction medium.

2. The method of claim 1, wherein the reaction medium is actively cooled to a temperature range from about 0° C. to about 25° C.

3. The method of claim 2, wherein the reaction medium is actively cooled to a temperature range from about 5° C. to about 17.5° C.

4. The method of claim 2, wherein the reaction medium is actively cooled to a temperature range from about 10° C. to about 17.5° C.

5. The method of claim 2, wherein the reaction medium is actively cooled to a temperature set point within the temperature range of from about 0° C. to about 25° C. and is maintained within 1° C. of the temperature set point throughout the deacylation reaction.

6. The method of claim 1, wherein the reaction medium is disposed in a reaction vessel, and wherein the reaction medium is actively cooled by cooling the reaction vessel.

7. The method of claim 1, wherein, upon raising the pH of the reaction medium to a pH of at least about 11, the deacylation reaction converts the sucralose-six-ester to sucralose to produce a sucralose yield that varies over time, wherein the deacylation reaction produces a maximum sucralose yield during a peak yield time, during which time the sucralose yield is at least substantially close to the maximum sucralose yield, followed by a reduction in the sucralose yield as the reaction continues, and wherein the predetermined temperature of the reaction medium and the pH of the reaction medium is selected to prolong the peak yield time.

8. The method of claim 7, wherein the peak yield time is prolonged to between about one hour and about three hours.

9. The method of claim 7, wherein neutralizing the pH of the reaction medium occurs during the peak yield time.

10. The method of claim 1, wherein the pH of the reaction medium is raised to a pH between about 12.5 and about 13.5, and wherein the temperature of the reaction medium is maintained at a temperature between about 5° C. and about 25° C.

11. The method of claim 10, wherein the temperature of the reaction medium is maintained at a temperature between about 10° C. and about 17.5° C.

12. The method of claim 1, wherein the sucralose-6-ester is selected from one or more of sucralose-6-acetate and sucralose-6-benzoate.

13. The method of claim 1, further comprising removing the tertiary amide from the reaction medium after neutralizing the pH of the reaction medium.

14. A method of deacylating sucralose-6-ester, comprising:
providing a reaction vessel containing a feed mixture of sucralose-six-ester, salt including alkaline metal or alkaline earth metal chloride, water, and other chlorinated sucralose byproducts in a reaction medium comprising a tertiary amide;
providing a heat exchange system thermally coupled to the reaction vessel;
controlling the heat exchange system to actively cool the reaction medium to a predetermined temperature range;
adding a base to the reaction medium to raise the pH of the reaction medium to a pH of at least about 11 while continuing to actively cool the reaction medium to maintain the temperature in the predetermined temperature range to initiate a deacylation reaction and to produce a solution comprising sucralose, salt including alkaline metal or alkaline earth metal chloride, water, and other chlorinated sucrose byproducts; and
neutralizing the pH of the reaction medium.

15. The method of claim 14, wherein actively cooling of the reaction medium includes circulating a heat transfer fluid through the heat exchange system.

16. The method of claim 15, wherein the reaction medium is actively cooled to a temperature range from about 0° C. to about 25° C.

17. The method of claim 16, wherein the reaction medium is actively cooled to a temperature range from about 5° C. to about 17.5° C.

18. The method of claim 17, wherein the reaction medium is actively cooled to a temperature range from about 10° C. to about 17.5° C.

19. The method of claim 15, wherein the reaction medium is actively cooled to a temperature set point within the temperature range of from about 0° C. to about 25° C. and is maintained within 1° C. of that temperature set point throughout the deacylation reaction.

20. The method of claim 14, wherein, upon raising the pH of the reaction medium to a pH of at least about 11, the deacylation reaction converts the sucralose-six-ester to sucralose to produce a sucralose yield that varies over time, wherein the deacylation reaction produces a maximum sucralose yield during a peak yield time, during which time the sucralose yield is at least substantially close to the maximum sucralose yield, followed by a reduction in the sucralose yield as the reaction continues, and wherein the predetermined temperature of the reaction medium and the pH of the reaction medium is selected to prolong the peak yield time.

21. The method of claim 20, wherein the peak yield time is prolonged to between about one hour and about three hours.

22. The method of claim 20, wherein neutralizing the pH of the reaction medium occurs during the peak yield time.

23. The method of claim 14, wherein the pH of the reaction medium is raised to a pH between about 12.5 and about 13.5, and wherein the temperature of the reaction medium is maintained at a temperature between about 5° C. and about 25° C.

24. The method of claim 23, wherein the temperature of the reaction medium is maintained at a temperature between about 10° C. and about 17.5° C.

25. The method of claim 14, wherein the sucralose-6-ester is selected from one or more of sucralose-6-acetate and sucralose-6-benzoate.

26. The method of claim 14, further comprising removing the tertiary amide from the reaction medium after neutralizing the pH of the reaction medium.
